(19) United States
(12) Patent Application Publication
(10) Pub. No.: US 2025/0149177 A1
(43) Pub. Date: May 8, 2025

(54) DEEP LEARNING BASED UNSUPERVISED DOMAIN ADAPTATION VIA A UNIFIED MODEL FOR MULTI-SITE PROSTATE LESION DETECTION

(71) Applicant: Siemens Healthineers AG, Forchheim (DE)

(72) Inventors: Bin Lou, Princeton Junction, NJ (US); Ali Kamen, Skillman, NJ (US)

(21) Appl. No.: 18/751,957

(22) Filed: Jun. 24, 2024

Related U.S. Application Data
(60) Provisional application No. 63/595,458, filed on Nov. 2, 2023.

(51) Int. Cl. G16H 50/30; G06T 7/00; G16H 30/40

(52) U.S. Cl. CPC G16H 50/30; G06T 7/0012; G16H 30/40; G06T 2207/20084; G06T 2207/30081; G06T 2207/30096

(57) ABSTRACT

Systems and method for performing a medical imaging analysis task via unsupervised domain adaptation are provided. 1) one or more input medical images of a patient and 2) one or more first image acquisition parameters associated with the one or more input medical images are received. One or more synthetic medical images associated with one or more second image acquisition parameters are generated. The one or more synthetic medical images are generated from at least one of the one or more input medical images using one or more machine learning based generator networks based on the one or more first image acquisition parameters. A medical imaging analysis task is performed using a machine learning based task network based on the one or more synthetic medical images. Results of the medical imaging analysis task are output.

### CROSS-REFERENCE TO RELATED APPLICATIONS

This application claims the benefit of U.S. Provisional Application No. 63/595,458, filed Nov. 2, 2023, the disclosure of which is herein incorporated by reference in its entirety.

### TECHNICAL FIELD

The present invention relates generally to prostate lesion detection in medical images, and in particular to deep learning based unsupervised domain adaptation via a unified model for multi-site prostate lesion detection.

### BACKGROUND

Prostate cancer is one of the most common cancers in males. Early detection of prostate cancer typically results in better treatment outcomes and lower mortality rates. Recently, machine learning methods have been proposed for prostate cancer detection on mpMRI (multiparametric magnetic resonance imaging) and bpMRI (biparametric magnetic resonance imaging) images. Such machine learning methods have been found to produce reliable results for in-domain test samples that are tightly matched to the training set. However, in real-world scenarios, clinicians often have their own preferences for b-value selection in mpMRI and bpMRI images. While variances in ADC and high b-value images due to diverse b-value selection may appear negligible to human observers, such variance can significantly influence results generated by machine learning methods. This is largely attributed to the domain shift observed across images from different datasets. The performance of machine learning methods drops when encountering domain shifts for processing out-of-distribution test samples whose b-values are not included in the training set.

### BRIEF SUMMARY OF THE INVENTION

In accordance with one or more embodiments, systems and method for performing a medical imaging analysis task via unsupervised domain adaptation are provided. 1) one or more input medical images of a patient and 2) one or more first image acquisition parameters associated with the one or more input medical images are received. One or more synthetic medical images associated with one or more second image acquisition parameters are generated. The one or more synthetic medical images are generated from at least one of the one or more input medical images using one or more machine learning based generator networks based on the one or more first image acquisition parameters. A medical imaging analysis task is performed using a machine learning based task network based on the one or more synthetic medical images. Results of the medical imaging analysis task are output.

In one embodiment, the one or more first image acquisition parameters and the one or more second image acquisition parameters comprise b-values. In one embodiment, the one or more first image acquisition parameters and the one or more second image acquisition parameters comprise at least one of field strength, signal-to-noise ratio, sequence selection, or a number of averages.

In one embodiment, the one or more machine learning based generator networks have a same architecture with different parameters.

In one embodiment, the one or more first image acquisition parameters are out-of-domain of training data on which the machine learning based task network was trained and the one or more second image acquisition parameters are in-domain of the training data on which the machine learning based task network was trained.

In one embodiment, the medical imaging analysis task is performed further based on remaining images of the one or more input medical images. The remaining images remain from the at least one of the one or more input medical images from which the one or more synthetic medical images are generated. In one embodiment, the one or more synthetic medical images are concatenated with the remaining images of the one or more input medical images. The medical imaging analysis task is performed based on the concatenated images.

In one embodiment, the one or more input medical images comprise a T2-weighted image, a diffusion-weighted imaging image, an apparent diffusion coefficient images, and an anatomical mask.

In one embodiment, the medical imaging analysis task comprises prostate cancer detection.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing a medical imaging analysis task via unsupervised domain adaptation, in accordance with one or more embodiments;

FIG. 2 shows a workflow for performing prostate cancer detection via unsupervised domain adaptation, in accordance with one or more embodiments;

FIG. 3 shows a workflow for training one or more machine learning based generator networks and a machine learning based task network for performing prostate cancer detection, in accordance with one or more embodiments;

FIG. 4 shows a table of case-level performance of embodiments described herein;

FIG. 5 shows a table of lesion-level performance of embodiments described herein;

FIG. 6 shows a table of results assessing embodiments described herein;

FIG. 7 shows exemplary images of original and synthetic images in accordance with embodiments described herein;

FIG. 8 shows an exemplary artificial neural network that may be used to implement one or more embodiments;

FIG. 9 shows a convolutional neural network that may be used to implement one or more embodiments;

FIG. 10 shows a data flow diagram of a generative adversarial network that may be used to implement one or more embodiments; and

FIG. 11 shows a high-level block diagram of a computer that may be used to implement one or more embodiments.

### DETAILED DESCRIPTION

The present invention generally relates to methods and systems for deep learning based unsupervised domain adaptation via a unified model for multi-site prostate lesion detection. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for unsupervised domain adaptation using a unified model to address domain shift and label availability for prostate cancer detection. Instead of multiple models, only a single unified model is utilized for unsupervised domain adaptation. Further, in order to achieve better performance of the unified model in multi-domain scenarios, a dynamic filter is utilized to leverage domain information. When benchmarked against conventional methods, the unsupervised domain adaptation in accordance with embodiments described herein consistently demonstrated an enhanced capability to perform prostate cancer detection more accurately.

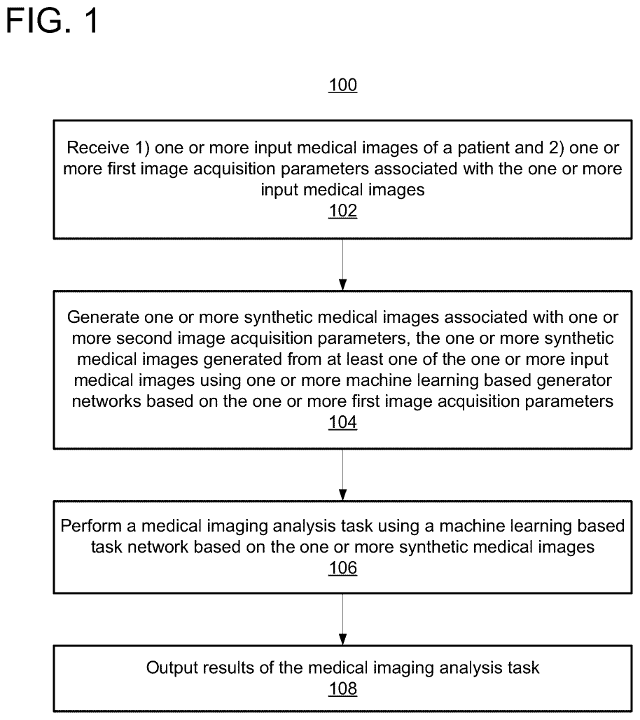

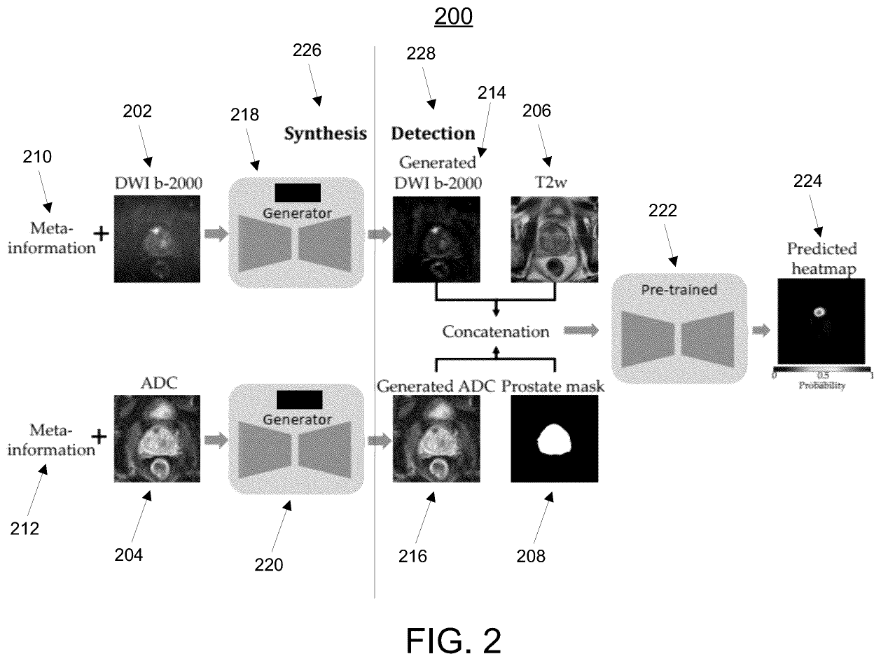

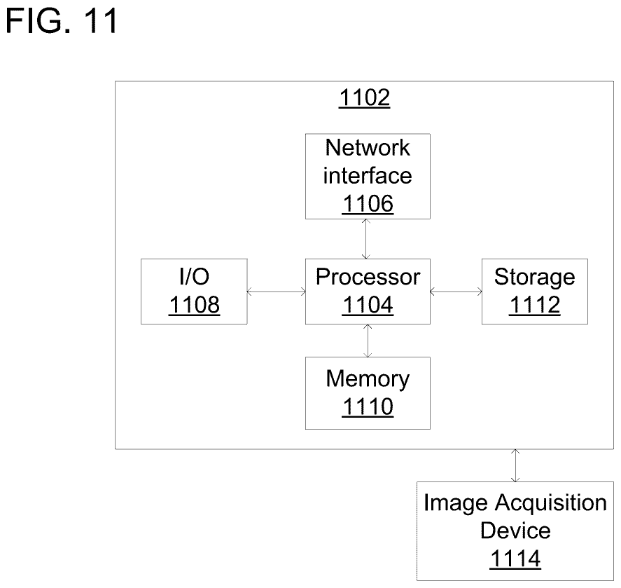

FIG. 1 shows a method 100 for performing a medical imaging analysis task via unsupervised domain adaptation, in accordance with one or more embodiments. The steps and sub-steps of method 100 may be performed by one or more suitable computing devices, such as, e.g., computer 1102 of FIG. 11. FIG. 2 shows a workflow 200 for performing prostate cancer detection via unsupervised domain adaptation, in accordance with one or more embodiments. Workflow 200 comprises a synthesis stage 226 and a detection stage 228. During synthesis stage 226, synthetic medical images associated with second image acquisition parameters are generated based on input medical images associated with first image acquisition parameters for domain adaptation. During detection stage 228, prostate cancer detection is performed based on the synthetic medical images. FIG. 1 and FIG. 2 will be described together.

At step 102 of FIG. 1, 1) one or more input medical images of a patient and 2) one or more first image acquisition parameters associated with the one or more input medical images are received. In one example, as shown in workflow 200 of FIG. 2, the one or more input medical images are medical images 202-208 and the one or more first image acquisition parameters are meta-information 210 and 212.

In one embodiment, the one or more input medical images depict one or more lesions on a prostate of the patient. For example, medical images 202-208 of FIG. 2 depict lesions on a prostate of a patient. However, the one or more input medical images may depict any other suitable anatomical objects of interest, such as, e.g., other organs, vessels, bones, tumors or other abnormalities, etc.

In one embodiment, the one or more input medical images are mpMRI or bpMRI images comprising, for example, T2W (T2-weighted), DWI (diffusion-weighted imaging), ADC (apparent diffusion coefficient) images/maps, and anatomical masks. For example, medical images 202-208 of FIG. 2 are DWI b-2000 image 202, ADC image 204, T2W image 206, and prostate mask 208 respectively. However, the one or more input medical images may be of any other suitable modality or modalities, such as MRI (magnetic resonance imaging), CT (computed tomography), US (ultrasound), x-ray, or any other medical imaging modality or combinations of medical imaging modalities. The first input medical image and/or the second input medical image may be 2D (two dimensional) images and/or 3D (three dimensional) volumes.

The one or more first image acquisition parameters may comprise any parameter for acquiring the one or more input medical images. In one embodiment, the one or more first image acquisition parameters are b-values. In the context of mpMRI and bpMRI images, b-values are parameters used in DWI to quantify the degree of diffusion weighting applied to the MRI images. Low b-values (e.g., generally 0 to 200 seconds per square millimeter) are less sensitive to diffusion and provide images with higher signal-to-noise ratio but less diffusion contrast while high b-values (e.g., generally 600 to 2000 seconds per square millimeter or higher) are highly sensitive to diffusion and provide strong diffusion contrast but with a lower signal-to-noise ratio. However, the one or more first image acquisition parameters may comprise any other suitable parameter for acquiring or generating the one or more input medical images, such as, e.g., field strength, signal-to-noise ratio, sequence selection, the number of averages, etc.

The one or more input medical images and/or the one or more first image acquisition parameters may be received, for example, by directly receiving the one or more input medical images from an image acquisition device (e.g., image acquisition device 1114 of FIG. 11) as the images are acquired, by loading the one or more input medical images and/or the one or more first image acquisition parameters from a storage or memory of a computer system (e.g., storage 1112 or memory 1110 of computer 1102 of FIG. 11), or by receiving the one or more input medical images and/or the one or more first image acquisition parameters from a remote computer system (e.g., computer 1102 of FIG. 11). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, one or more synthetic medical images associated with one or more second image acquisition parameters are generated. The one or more synthetic medical images are generated from at least one of the one or more input medical images using one or more machine learning based generator networks based on the one or more first image acquisition parameters.

The one or more second image acquisition parameters may comprise the same parameters as the one or more first image acquisition parameters but with different values. In one embodiment, the one or more second image acquisition parameters are image acquisition parameters with standard values that are in-domain of the training data used for training the machine learning based task network utilized at step 106 of FIG. 1. For example, where the one or more first image acquisition parameters comprise b-values, the one or more second image acquisition parameters may comprise b-values that are in the PI-RADS (prostate imaging reporting and data system) standard range, i.e., having b-values between 0-100 sec/mm² (preferably 50-100 sec/mm²) for low b-values and b-values between 800-1000 sec/mm² for intermediate b-values.

The one or more machine learning based generator networks may be implemented according to any suitable machine learning based architecture for generating the one or more synthetic medical images. In one embodiment, the one or more machine learning based generator networks are U-Net based generator networks. The one or more machine learning based generator networks comprise a respective generator network for each of the one or more synthetic medical images. For example, as shown in FIG. 2, workflow 200 comprises generator 218 for generating generated DWI b-2000 image 214 from DWI b-2000 image 202 and generator 220 for generating generated ADC image 216 from ADC image 204. Each of the one or more generator networks are implemented with the same architecture but weighted with different parameters, thus providing for a unified model. Each of the one or more generators receives as input 1) one of the one or more input medical images and 2) an associated one of the one or more first image acquisition parameters and generates as output one of the one or more synthetic medical images.

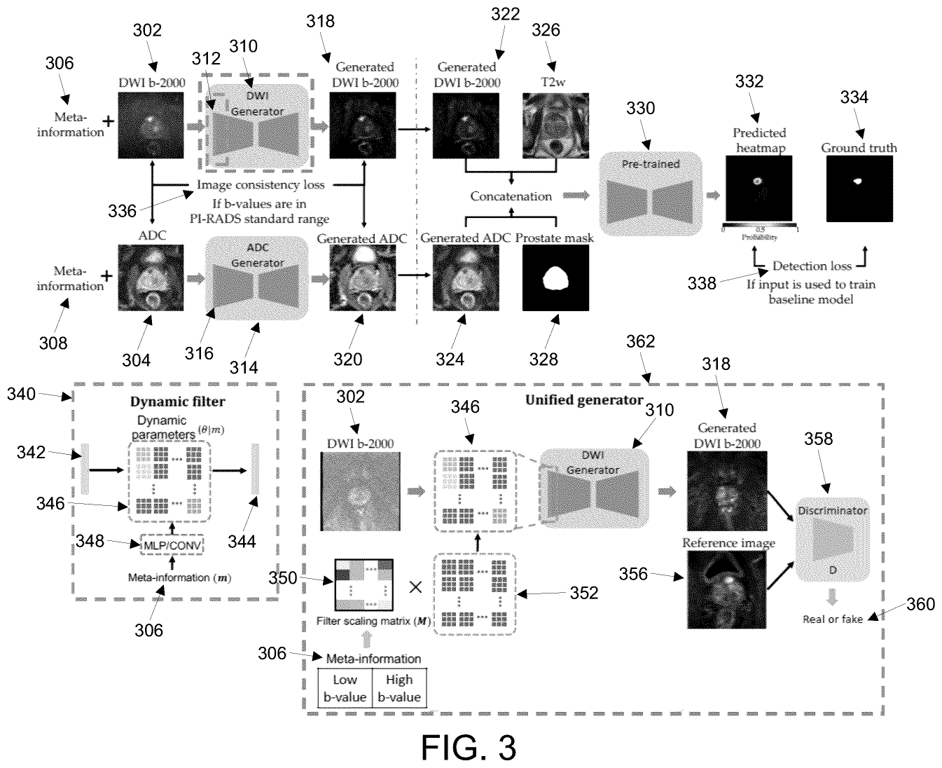

The one or more machine learning based generator networks are trained during a prior offline or training stage. In one embodiment, the one or more machine learning based generator networks are trained according to workflow 300 of FIG. 3. Once trained, the one or more machine learning based generator networks are applied during an online or inference stage, e.g., to perform step 104 of FIG. 1.

At step 106 of FIG. 1, a medical imaging analysis task is performed using a machine learning based task network based on the one or more synthetic medical images. In one embodiment, the medical imaging analysis task is prostate cancer detection. However, the medical imaging analysis task may be any other suitable medical imaging analysis task, such as, e.g., segmentation, detection, classification, quantification, image generation, etc.

In one embodiment, the medical imaging analysis task is performed further based on remaining ones of the one or more input medical images (that remain from the at least one of the one or more input medical images from which the one or more synthetic medical images are generated at step 104 of FIG. 1). For example, as shown in workflow 200 of FIG. 2, prostate cancer detection is performed based on generated DWI b-2000 images 214 and generated ADC image 216 as well as the remaining T2W image 206 and prostate mask 208.

The machine learning based task network may be implemented according to any suitable machine learning based architecture for performing the medical imaging analysis task. In one embodiment, the machine learning based task network is a pre-trained U-Net based task network. The machine learning based task network receives as input the one or more synthetic medical images and, optionally, the remaining ones of the one or more input medical images and generates as output results of the medical imaging analysis task. For example, as shown in workflow 200 of FIG. 2, the synthetic images (generated DWI b-2000 image 214 and generated ADC image 216) are combined (e.g., concatenated) with the remaining input medical images (T2W image 206 and prostate mask 208) and task network 222 receives as input the combined images and generates as output a predicted heatmap 224 identifying prostate cancer lesions.

The machine learning based task network is trained during a prior offline or training stage. In one embodiment, the machine learning based task network is trained according to workflow 300 of FIG. 3. Once trained, the machine learning based task network is applied during an online or inference stage, e.g., to perform step 106 of FIG. 1.

At step 108 of FIG. 1, results of the medical imaging analysis task are output. For example, the results of the medical imaging analysis task can be output by displaying the results on a display device of a computer system (e.g., I/O 1108 of computer 1102 of FIG. 11), storing the results on a memory or storage of a computer system (e.g., memory 1110 or storage 1112 of computer 1102 of FIG. 11), or by transmitting the results to a remote computer system (e.g., computer 1102 of FIG. 11).

FIG. 3 shows a workflow 300 for training one or more machine learning based generator networks and a machine learning based task network for performing prostate cancer detection (or any other medical imaging analysis task), in accordance with one or more embodiments. In one example, generators 310 and 314 are the one or more machine learning based generator networks utilized at step 104 of FIG. 1 and task network 330 is the machine learning based task network utilized at step 106 of FIG. 1. Workflow 300 is performed during an offline or training stage to train generators 310 and 314 and task network 330. Once trained, generators 310 and 314 and task network 330 may be applied during an online or testing stage, e.g., to respective perform steps 104 and 106 of FIG. 1.

Workflow 300 is performed using training images 302, 304, 326, and 328 along with meta-information 306 and 308. Training images 302, 304, 326, and 328 comprise DWI b-2000 image 302, ADC image 304, T2W image 326, and prostate mask 329 as part of mpMRI or bpMRI imaging. Meta-information 306 and 308 comprises image acquisition parameters (e.g., b-values) respectively associated with DWI b-2000 image 302 and ADC image 304. Generator 310 receives as input 2D DWI b-2000 image 302 from the target domain and meta-information 306 and generates as output 2D generated (or synthetic) DWI b-2000 image 318 styled in the reference domain. Generator 314 receives as input 2D ADC image 304 and meta-information 308 and generates as output generated (or synthetic) ADC image 320. Generators 310 and 314 may be implemented according to a U-shaped network. While the architectures of generators 310 and 314 are identical, they are weighted with different parameters (i.e., separate networks are trained for the DWI b-2000 images and the ADC images).

An exploded view 362 of the workflow for training generator 310 is shown in FIG. 3. Generator 314 is similarly trained. Discriminator 358 evaluates the performance of generator 310 during training by distinguishing between generated DWI b-2000 image 318 and real reference image 356 to determine whether the generated DWI b-2000 image 318 is real or fake 360. When training images 302, 304, 326, and 328 and their ground truth labels 334 are used for training, a detection loss 338 is applied to offer supplementary guidance to the generator 310. To emphasize correct mapping to the reference domain, an additional consistency loss L_{consistency} 336 is employed at the image level to preserve the original information from the input images, especially if its b-values align with the PI-RADS standard range. In one embodiment, consistency loss L_{consistency} 336 is the mean square error.

Unlike conventional methods which require multiple networks for multiple domain mappings, embodiments described herein employ only a unified model for each modality. However, the performance of a unified model may be limited in producing robust results between multiple domains due to lack of domain information. To address this issue, dynamic filters 312 and 316 are utilized as a domain indicator for generators 310 and 314 respectively. An exploded view 340 of dynamic filter 312 is shown in FIG. 3. Dynamic filter 316 is similarly implemented. Dynamic filters 312 and 316 aim to increase domain generalizability of generators 310 and 314 by leveraging meta-information 306 and 308 respectively. As shown in exploded view 340 of FIG. 3, parameters 346 of dynamic filter 312 are dynamically generated between feature maps 342 and 344 of generator 310 based on various different conditions by applying a scaling (e.g., MLP (multi-layer perceptron)/Convolutional) layer 348 to meta-information 306. As shown in exploded view 362, low and high b-values from meta-information 306 are converted into a 2D tensor, which simplifies the input for the domain controller and preserves original information. A filter scaling strategy generates a kernel-wise scale factor, uniformly weighting all parameters instead of individually scaling them. Specifically, the domain controller learns to generate the corresponding filter scaling matrix M 350 based on meta-information 306. Each element in filter scaling matrix M 350 represent the scale of the corresponding kernel, and the parameters 346 of the dynamic filter are dynamically adjusted through scalar multiplication of parameters 352.

Task network 330 is implemented as a 2D detection network for prostate cancer detection in workflow 300. The detection network may be implemented as a U-Net embedded with residual blocks. Task network 330 receives as input a concatenation of generated DWI b-2000 image 322, generated ADC image 324, T2W image 326, and prostate mask 328 and generates as output a predicted heatmap 332 where non-zero regions are considered as lesion candidates. It should be understood that while generated DWI b-2000 image 322 and generated DWI b-2000 image 318 and generated ADC image 324 and generated ADC image 320 are separately shown in workflow 300 for ease of illustration, generated DWI b-2000 image 322 and generated DWI b-2000 image 318 are the same image and generated ADC image 324 and generated ADC image 320 are the same image. Predicted heatmap 332 is compared with ground truth labels 334 via detection loss 338. To identify the TPs (true positives) and FPs (false positives), a threshold is used to get a set of connected components. The TPs can be identified if the connected components overlap on annotations or are less than 5 millimeters away from the lesion center. Otherwise, such connected components are classified as FPs. Any lesions that lack corresponding detections are terms FNs (false negatives)

Embodiments described herein were experimentally validated. A multi-cohort dataset of 5,150 cases from nine different clinical sites were used, all of which have bpMRI prostate examinations consisting of T2-weighted (T2w) acquisition and DWI of at least two different b-values. The inclusion criteria for the experimental validation were as follows: (1) patients who were treatment-naïve; (2) clear visibility of the prostate gland in the field of view of bi-parametric MRI images; (3) acquisition of images using 1.5 T or 3 T axial MRI scans with either a body or endorectal coil. Conversely, the exclusion criteria were: (1) cases involving prostatectomy or any scenario where the prostate was partially resected; and (2) cases with severe artifacts resulting from implants or motion. In this experimental validation, all qualified b-values were included to investigate their impact on the detection performance. The b-values ranging from 0 to 200 were considered as low b-values, while those between 600 and 2000 were considered as high b-values. The ADC images and DWI b-2000 images were computed based on each pair of low b-value and high b-value DWI images by performing a nonlinear least-squares fit to the equation S(b)=S₀·e^{-b·ADC}. For each voxel, the coefficient of b was employed as its ADC value (with a scaling factor of 10⁶), and the intensity of b-2000 images was calculated through extrapolation at b=2000. To maintain consistency and reduce variation in ADC computation, vendor-provided ADC maps were excluded. In this way, each pair of b-values from the same case can be considered as a unique sample from a different domain. This yielded a total of 14,191 samples of 34 different combinations of b-values from all cases. All samples were categorized into a few subgroups based on the range of b-values.

A total of 3,458 cases were used for training. For training the baseline supervised learning method, the best pair of b-values (optimal) was selected and only one single sample from each case was used. PI-RADS guidelines recommend using one low b-value set at 0-100 sec/mm² (preferably 50-100 sec/mm²) and one intermediate b-value set at 800-1000 sec/mm² for ADC computations. Accordingly, the b-values that are the closest to 50 and 1000 were selected as low and high b-values, respectively. For other methods (generic model and the proposed unsupervised domain adaptation methods), additional samples with all possible b-value pairs were used, which consisted of 11,763 samples from the same training cases. In the unsupervised domain adaptation training process, 882 samples whose b-values are from the standard domain (low b-value=50, high b-value=800) were selected as reference domain data to train the unified generator of unsupervised domain adaptation methods, and the rest of the data were considered target domain samples. The independent testing set contained 1,692 cases with 2,428 samples. The results of 2,393 samples are reported due to very limited sample number for some b-value subgroups. All the cases had lesion-based PI-RADS information and voxel-based annotations of the lesion boundaries. The prostate cancer lesion annotations were obtained based on the clinical radiology reports and carefully reviewed by an expert radiologist (DW) with five years of experience in radiology, specializing in prostate MRI examinations. A positive case was identified if it contained PI-RADS≥3 lesions.

PI-RADS guidelines recommends a high b-value set at ≥1,400 sec/mm². Similarly, a high b-value image was recomputed at a fixed b=2,000 sec/mm² to ensure a representation in which lesions stand out. The fixed b-value was selected to further eliminate the b-value variances among datasets. The image preprocessing pipeline took raw bpMRI acquisitions and generated well-formatted data volumes for all subsequent synthesis and detection models.

Embodiments described herein were applied for prostate cancer detection to solve two common practical issues: domain shift and label availability for test data. The proposed framework comprises two parts: synthesis and detection. To increase the generalizability of the detection network for out-of-distribution test samples, generators align the style of DWI B-2000 and ADC test samples from the target domain to the reference domain at the image level. Next, the detection model predicts the prostate cancer heatmap which uses the concatenation of T2w, generated ADC, generated DWI B-2000, and prostate mask as inputs. Notably, this entire process operates without the need for test data labels. To more accurately mimic real-world scenarios, the detection model was initially trained and then the trained model was used to educate the generators.

The training process of the detection model (baseline) used binary cross-entropy as detection loss (L_{det}). To train the generator, the batch size was set to 96 and a loss function L_{CUT} was used. The total epoch was set to 100. The domain controller is a simple convolutional layer with 7 as kernel size, an input channel of 2, and an output channel of 128, where 64 scaling factors and bias weights are included. In addition, the loss selections of generators (L_{syn}) depend on three scenarios related to the input image: (1) L_{syn}=L_{CUT} for unlabeled target domain data; (2) L_{syn}=L_{det}+L_{CUT} for labeled target domain data; and (3) L_{syn}=L_{det}+L_{CUT}+L_{consistency} for reference domain data.

The proposed framework was compared with two conventional deep learning methods for prostate cancer lesion detection using multisite bpMRI datasets, which are (1) baseline: a supervised learning pre-trained detection model; and (2) a generic model: retrain the baseline method using a larger dataset with various b-values.

The area under the receiver operating characteristic curve (AUC) score was computed as case-level performance, which is the primary endpoint. The maximum value of the 3D heatmap was defined as the prediction score of the sample to calculate the AUC score. The confidence interval was computed based on a bootstrap approach with 2,000 resamples. The statistical results were conducted with Python with numpy, sklearn and scipy libraries. A statistical significance threshold of 0.05 was set. In addition, the free-response receiver operating characteristic (FROC) was used as a metric to evaluate the lesion-level performance as supplementary results. Moreover, peak signal-to-noise ratio (PSNR), mean square error (MSE), and structural similarity index measure (SSIM) were used as metrics to evaluate the image quality of generated images. This requires the same cases to have DWI images in both reference domain and other target domains. To achieve this, all cases from the testing dataset that had six different b-values were selected. The DWI B-2000 images were computed using naturally acquired DWI images of three different b-value pairs: (50, 800), (150, 1500) and (200, 2000). The proposed method was applied to the B-2000 images computed using b-values of (150, 1500) and (200, 2000) to generate new B-2000 images. The original and generated B-2000 images were compared with the corresponding one computed by using (50, 800) b-values. t-SNE (t-distributed stochastic neighbor embedding) visualization was used to assess the impact of the generated ADC and DWI B-2000 images on the detection network. Specifically, 100 samples from the unseen test set with a low b-value of 200 and a high b-value of 2000 were randomly selected. These samples served as input for the proposed framework. For the t-SNE visualization, the feature maps of these selected cases were extracted from the bottleneck feature map of the baseline method.

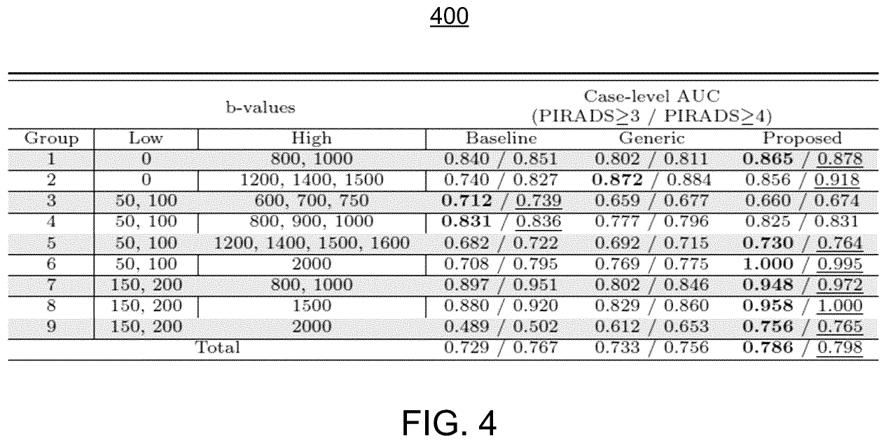

Case Level Performance: Table 400 of FIG. 4 shows the case-level performance of the proposed approach in accordance with embodiments described herein compared with conventional baseline and generic approaches. For both PI-RADS≥3 and PI-RADS>4 labels, the overall performance of the proposed model was significantly higher (p<0.001) compared with other models. The baseline method was effective when test data b-values were in-domain (i.e., the test data b-values closely matched with the training set), primarily in the reference domain (group 4), but struggled with out-of-distribution samples, as seen in group 9. Even with diverse b-values added to the training data, the generic model failed to differentiate across domains, resulting in unstable performance, especially in the commonly used reference domain. The proposed approach improved the results of the baseline method in all groups when b-values deviated from the standard and maintained consistent performance for the reference domain.

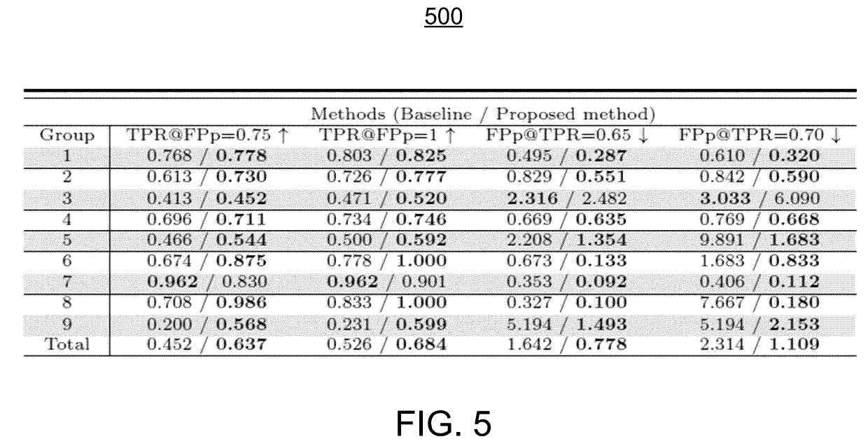

Lesion Level Performance: FROC values representing the FP to TP ratio are shown in Table 500 of FIG. 5. The proposed method outperformed the baseline in most domains except for the FP rate in group 3 and the TP rate in group 7. For standard b-values, the baseline method had higher AUC scores; but the proposed method demonstrates higher overall performance, particularly when b-values diverged from the standard.

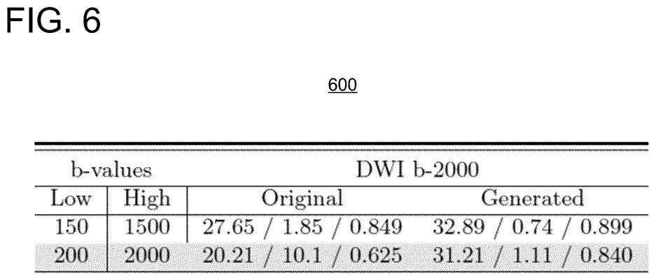

Quality of Generated Images: The proposed framework was assessed in terms of the image quality of generated DWI B-2000 images through comparison with paired DWI B-2000 images in the reference domain. The PSNR (peak signal-to-noise ratio), MSE (mean squared error), and SSIM (structural similarity) index results are provided in table 600 of FIG. 6. In the analysis of both b-value pairs, the generated images had higher PSNR, lower MSE and higher SSIM. The results indicate that the DWI B-2000 images generated by our method are more similar to the reference domain images than the original target domain images.

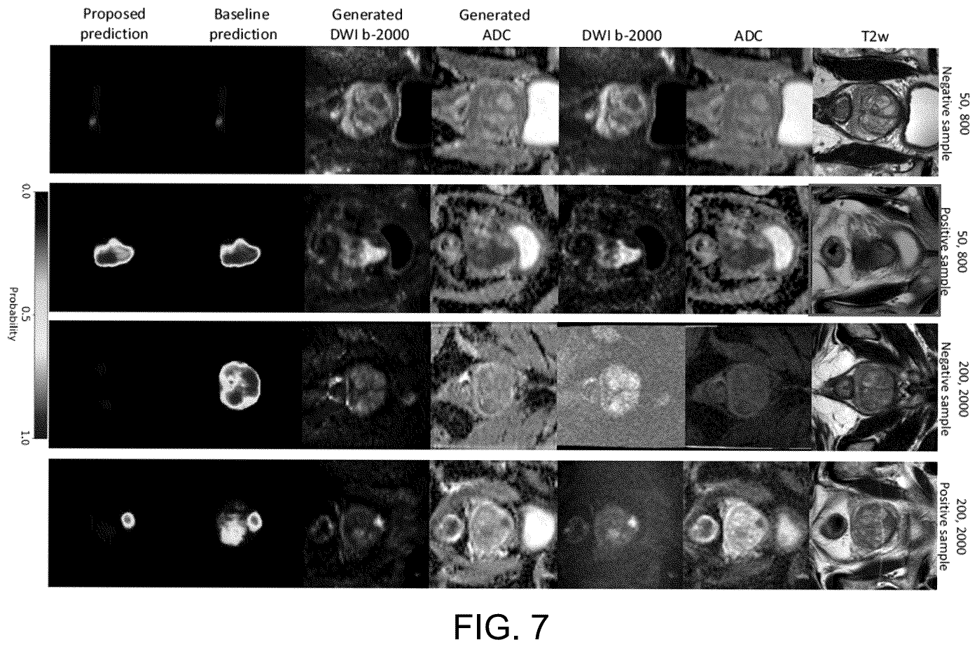

FIG. 7 shows images 700 comprising original bpMRI images, generated DWI images, and detection heatmaps of 4 example cases (2 positive and 2 negative). In each group, one case is from the reference domain and one case is from the target domain with b-values far from the PI-RADS guideline recommendation. For cases in the reference domain, no obvious changes were observed in the generated images, and the predictions were similar by using original or generated images. For cases not in the reference domain, a significant improvement in image quality was observed for the generated images. The predictions were also more accurate when compared with the reference standard annotations.

T-SNE Visualization: To better visualize the relationship between reference domain images, original target domain images and generated images, the t-SNE algorithm was applied to reduce the bottleneck layer features of the prostate cancer detection network to a 2-dimensional representation. For neat visualization, 100 cases whose original DWI acquisitions had low b-value=200 and high b-value=2000 were selected for this analysis. The original and generated DWI images were used as input of the detection network. Another 100 cases from the PROSTATEx Challenge dataset were selected as the reference domain (i.e., low b-value=50 and high b-value=800) cases for comparison. The generated DWI images using the proposed method formed a tighter cluster compared with the original target domain image. Moreover, the generated images aligned more closely with the reference domain data, which indicates a higher similarity in the latent space of the detection network.

The novel unsupervised domain adaptation method was proposed with a unified model to solve practical common issues, specifically domain shift and label availability, for prostate cancer lesion detection. Only a unified model is used in the unsupervised domain adaptation method for multi-domain mapping instead of multiple networks being trained as in conventional methods. To achieve better performance of a unified model in multi-domain scenarios, a dynamic filter is utilized to leverage domain information. When benchmarked against other methods using a large-scale, multisite dataset comprising 5,150 cases (14,191 samples), the unsupervised domain adaptation method in accordance with embodiments described herein consistently demonstrated an enhanced capability to perform more accurate prostate cancer detection.

To demonstrate the feasibility for practical use, the experimental validation was conducted on a large-scale dataset with different imaging protocols, where the heterogeneous domain shifts are present and pose a challenge to achieve a consistent performance. The proposed unsupervised domain adaptation method leverages information from the entire dataset, notably unlabeled data, to reduce the annotation effort, which is usually a burden for large datasets. Importantly, the proposed unsupervised domain adaptation method can be seamlessly integrated into any pre-trained prostate cancer detection framework and can be used as an image adapter at the upstream level to reduce discrepancies between domains. The proposed unsupervised domain adaptation method overall improved the generalizability of downstream prostate cancer detection models. Importantly, there is no need to retrain or modify the network for new target data, making the method suitable for a variety of medical image applications.

To the knowledge of the inventors, no prior study has explored the domain shift in prostate cancer detection using ADC and DWI high b-value images, especially given the various domains in the experimental validation. The common practical solutions were validated as part of the experimental validation. Although such methods are not the optimal solutions, important findings emerged: (1) using the original test image is preferable if its b-values closely align with training set, and (2) retraining a generic model could produce unpredictable results due to its broad adaptability and limitations in specific learning.

Several existing studies have tried to address these prevalent practical challenges by producing more consistent DWI images. For instance, one existing study suggested recalculating ADC maps and high b-value images at a fixed 2000 sec/mm² rather than using the originally acquired images. However, this method cannot avoid the diffusion kurtosis effect if the acquired DWI uses a high b-value over 1500 sec/mm². The proposed unsupervised domain adaptation method with an image-to-image technique effectively translates out-of-domain target samples into the style of reference domain. Resulting generated ADC and DWI b-2000 images are similar to the real reference domain image both at image-level and latent-level. The most pronounced improvements occur when the high b-value deviates farther from the standard range. Additionally, when high b-values are within the reference domain, the proposed unsupervised domain adaptation method boosts performance for low b-values that are out-of-distribution samples. Comprehensive detection results indicate that high b-values influence domain discrepancy more than low b-values.

Embodiments described herein introduce a dynamic filter, which can be treated as a domain indicator and plugged it into any generator to leverage meta-information. The proposed dynamic filter generates conditional parameters according to the corresponding meta-information to differentiate domains. This is unlike encoding meta-information as one-hot vectors, which often requires a codebook to rigidly encode the corresponding relationship. Such an encoding approach might not be ideal for large-scale studies involving multiple domains. Additionally, the codebook would need adjustments whenever a new combination of b-values emerges. In contrast, an effective yet straightforward strategy has been proposed that directly uses b-values as input. This not only retains the original meta-information but also simplifies the process, making it adaptable to arbitrary b-value combinations.

In conclusion, the proposed unified model-based unsupervised domain adaptation method for prostate cancer detection showed marked improvement compared with the baseline model on a large multisite dataset, especially outside the reference domain. To the best of the inventors' knowledge, this is the first large-scale study exploring the impact of b-value properties on ADC and DWI b-2000 images with the aim of improving detection outcomes for a multi-domain scenario.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the one or more machine learning based generator networks utilized at step 104 or the machine learning based task network utilized at step 106 of FIG. 1, generators 218 and 220 and task network 222 of FIG. 2, generators 310 and 314 and task network 330 of FIG. 3, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

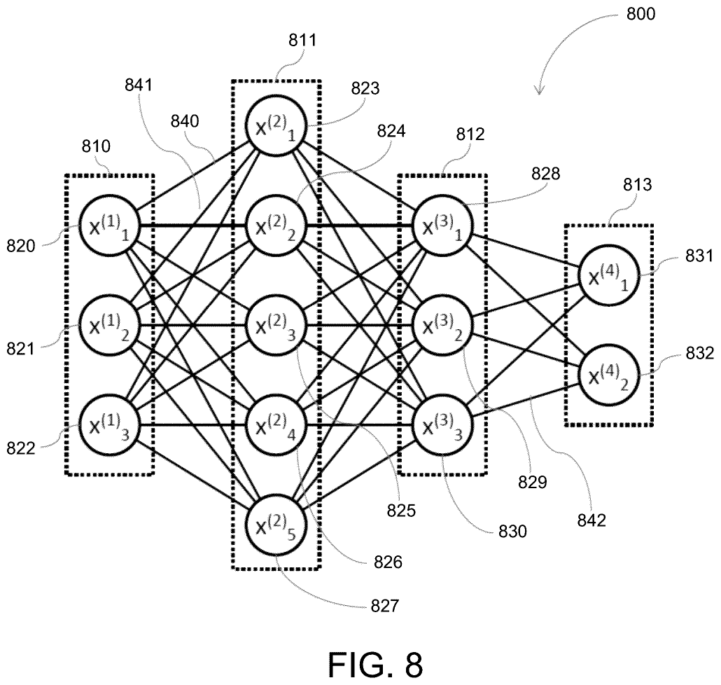

FIG. 8 shows an embodiment of an artificial neural network 800 that may be used to implement one or more machine learning models described herein. Alternative terms for “artificial neural network” are “neural network”, “artificial neural net” or “neural net”.

The artificial neural network 800 comprises nodes 820, . . . , 832 and edges 840, . . . , 842, wherein each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832, it is also possible that the first node 820, . . . , 832 and the second node 820, . . . , 832 are identical. For example, in FIG. 8 the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as “ingoing edge” for the second node 820, . . . , 832 and as “outgoing edge” for the first node 820, . . . , 832.

In this embodiment, the nodes 820, . . . , 832 of the artificial neural network 800 can be arranged in layers 810, . . . , 813, wherein the layers can comprise an intrinsic order introduced by the edges 840, . . . , 842 between the nodes 820, . . . , 832. In particular, edges 840, . . . , 842 can exist only between neighboring layers of nodes. In the displayed embodiment, there is an input layer 810 comprising only nodes 820, . . . , 822 without an incoming edge, an output layer 813 comprising only nodes 831, 832 without outgoing edges, and hidden layers 811, 812 in-between the input layer 810 and the output layer 813. In general, the number of hidden layers 811, 812 can be chosen arbitrarily. The number of nodes 820, . . . , 822 within the input layer 810 usually relates to the number of input values of the neural network, and the number of nodes 831, 832 within the output layer 813 usually relates to the number of output values of the neural network.

In particular, a (real) number can be assigned as a value to every node 820, . . . , 832 of the neural network 800. Here, x⁽ⁿ⁾ᵢ denotes the value of the i-th node 820, . . . , 832 of the n-th layer 810, . . . , 813. The values of the nodes 820, . . . , 822 of the input layer 810 are equivalent to the input values of the neural network 800, the values of the nodes 831, 832 of the output layer 813 are equivalent to the output value of the neural network 800. Furthermore, each edge 840, . . . , 842 can comprise a weight being a real number, in particular, the weight is a real number within the interval [−1, 1] or within the interval [0, 1]. Here, w^{(m,n)}_{i,j} denotes the weight of the edge between the i-th node 820, . . . , 832 of the m-th layer 810, . . . , 813 and the j-th node 820, . . . , 832 of the n-th layer 810, . . . , 813. Furthermore, the abbreviation w⁽ⁿ⁾_{i,j} is defined for the weight w^{(n,n+1)}_{i,j}.

In particular, to calculate the output values of the neural network 800, the input values are propagated through the neural network. In particular, the values of the nodes 820, . . . , 832 of the (n+1)-th layer 810, . . . , 813 can be calculated based on the values of the nodes 820, . . . , 832 of the n-th layer 810, . . . , 813 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 810 are given by the input of the neural network 800, wherein values of the first hid-den layer 811 can be calculated based on the values of the input layer 810 of the neural network, wherein values of the second hidden layer 812 can be calculated based in the values of the first hidden layer 811, etc.

In order to set the values w^{(m,n)}_{i,j} for the edges, the neural network 800 has to be trained using training data. In particular, training data comprises training input data and training output data (denoted as tᵢ). For a training step, the neural network 800 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 800 (backpropagation algorithm). In particular, the weights are changed according to

$${{w}^{′}}^{{( n )}_{i , j}} = {w}^{{( n )}_{i , j}} - y \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 813, wherein f′ is the first derivative of the activation function, and t⁽ⁿ⁺¹⁾ⱼ is the comparison training value for the j-th node of the output layer 813.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

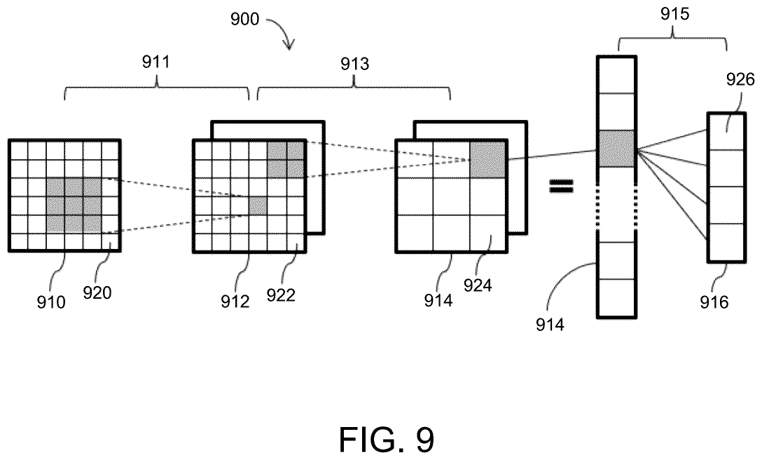

FIG. 9 shows an embodiment of a convolutional neural network 900 that may be used to implement one or more machine learning models described herein. In the displayed embodiment, the convolutional neural network comprises 900 an input node layer 910, a convolutional layer 911, a pooling layer 913, a fully connected layer 914 and an output node layer 916, as well as hidden node layers 912, 914. Alternatively, the convolutional neural network 900 can comprise several convolutional layers 911, several pooling layers 913 and several fully connected layers 915, as well as other types of layers. The order of the layers can be chosen arbitrarily, usually fully connected layers 915 are used as the last layers before the output layer 916.

In particular, within a convolutional neural network 900 nodes 920, 922, 924 of a node layer 910, 912, 914 can be considered to be arranged as a d-dimensional matrix or as a d-dimensional image. In particular, in the two-dimensional case the value of the node 920, 922, 924 indexed with i and j in the n-th node layer 910, 912, 914 can be denoted as x(n)[i, j]. However, the arrangement of the nodes 920, 922, 924 of one node layer 910, 912, 914 does not have an effect on the calculations executed within the convolutional neural network 900 as such, since these are given solely by the structure and the weights of the edges.

A convolutional layer 911 is a connection layer between an anterior node layer 910 (with node values x(n−1)) and a posterior node layer 912 (with node values x(n)). In particular, a convolutional layer 911 is characterized by the structure and the weights of the incoming edges forming a convolution operation based on a certain number of kernels. In particular, the structure and the weights of the edges of the convolutional layer 911 are chosen such that the values x(n) of the nodes 922 of the posterior node layer 912 are calculated as a convolution x(n)=K*x(n−1) based on the values x(n−1) of the nodes 920 anterior node layer 910, where the convolution * is defined in the two-dimensional case as

$${x}_{k}^{( n )} [ i , j ] = ( K * {x}^{( n - 1 )} ) [ i , j ] = {\sum }_{{i}^{1}} {\sum }_{{j}^{1}} K [ {i}^{1} , {j}^{1} ] \cdot {x}^{( n - 1 )} [ i - {i}^{1} , j - {j}^{1} ] .$$

Here the kernel K is a d-dimensional matrix (in this embodiment, a two-dimensional matrix), which is usually small compared to the number of nodes 920, 922 (e.g., a 3×3 matrix, or a 5×5 matrix). In particular, this implies that the weights of the edges in the convolution layer 911 are not independent, but chosen such that they produce said convolution equation. In particular, for a kernel being a 3×3 matrix, there are only 9 independent weights (each entry of the kernel matrix corresponding to one independent weight), irrespectively of the number of nodes 920, 922 in the anterior node layer 910 and the posterior node layer 912.

In general, convolutional neural networks 900 use node layers 910, 912, 914 with a plurality of channels, in particular, due to the use of a plurality of kernels in convolutional layers 911. In those cases, the node layers can be considered as (d+1)-dimensional matrices (the first dimension indexing the channels). The action of a convolutional layer 911 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 910, x⁽ⁿ⁾^{_{b}} corresponds to the b-th channel of the posterior node layer 912 and K_{a,b} corresponds to one of the kernels. If a convolutional layer 911 acts on an anterior node layer 910 with A channels and outputs a posterior node layer 912 with B channels, there are A·B independent d-dimensional kernels K_{a,b}.

In general, in convolutional neural networks 900 activation functions are used. In this embodiment re ReLU (acronym for “Rectified Linear Units”) is used, with R(z)=max(0, z), so that the action of the convolutional layer 911 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tan h or Softmax.

In the displayed embodiment, the input layer 910 comprises 36 nodes 920, arranged as a two-dimensional 6×6 matrix. The first hidden node layer 912 comprises 72 nodes 922, arranged as two two-dimensional 6×6 matrices, each of the two matrices being the result of a convolution of the values of the input layer with a 3×3 kernel within the convolutional layer 911. Equivalently, the nodes 922 of the first hidden node layer 912 can be interpreted as arranged as a three-dimensional 2×6×6 matrix, wherein the first dimension correspond to the channel dimension.

The advantage of using convolutional layers 911 is that spatially local correlation of the input data can exploited by enforcing a local connectivity pattern between nodes of adjacent layers, in particular by each node being connected to only a small region of the nodes of the preceding layer.

A pooling layer 913 is a connection layer between an anterior node layer 912 (with node values x(n−1)) and a posterior node layer 914 (with node values x(n)). In particular, a pooling layer 913 can be characterized by the structure and the weights of the edges and the activation function forming a pooling operation based on a non-linear pooling function f. For example, in the two-dimensional case the values x(n) of the nodes 924 of the posterior node layer 914 can be calculated based on the values x(n−1) of the nodes 922 of the anterior node layer 912 as

$${x}^{{( n )}_{b}} [ i , j ] = f \{ {x}^{( n - 1 )} [ {id}_{1} , {id}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 913 the number of nodes 922, 924 can be reduced, by re-placing a number d1·d2 of neighboring nodes 922 in the anterior node layer 912 with a single node 922 in the posterior node layer 914 being calculated as a function of the values of said number of neighboring nodes. In particular, the pooling function f can be the max-function, the average or the L2-Norm. In particular, for a pooling layer 913 the weights of the incoming edges are fixed and are not modified by training.

The advantage of using a pooling layer 913 is that the number of nodes 922, 924 and the number of parameters is reduced. This leads to the amount of computation in the network being reduced and to a control of overfitting.

In the displayed embodiment, the pooling layer 913 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 900 are fully connected layers 915. A fully connected layer 915 is a connection layer between an anterior node layer 914 and a posterior node layer 916. A fully connected layer 913 can be characterized by the fact that a majority, in particular, all edges between nodes 914 of the anterior node layer 914 and the nodes 916 of the posterior node layer are present, and wherein the weight of each of these edges can be adjusted individually.

In this embodiment, the nodes 924 of the anterior node layer 914 of the fully connected layer 915 are displayed both as two-dimensional matrices, and additionally as non-related nodes (indicated as a line of nodes, wherein the number of nodes was reduced for a better presentability). This operation is also denoted as “flattening”. In this embodiment, the number of nodes 926 in the posterior node layer 916 of the fully connected layer 915 smaller than the number of nodes 924 in the anterior node layer 914. Alternatively, the number of nodes 926 can be equal or larger.

Furthermore, in this embodiment the Softmax activation function is used within the fully connected layer 915. By applying the Softmax function, the sum the values of all nodes 926 of the output layer 916 is 1, and all values of all nodes 926 of the output layer 916 are real numbers between 0 and 1. In particular, if using the convolutional neural network 900 for categorizing input data, the values of the output layer 916 can be interpreted as the probability of the input data falling into one of the different categories.

In particular, convolutional neural networks 900 can be trained based on the backpropagation algorithm. For preventing overfitting, methods of regularization can be used, e.g., dropout of nodes 920, . . . , 924, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

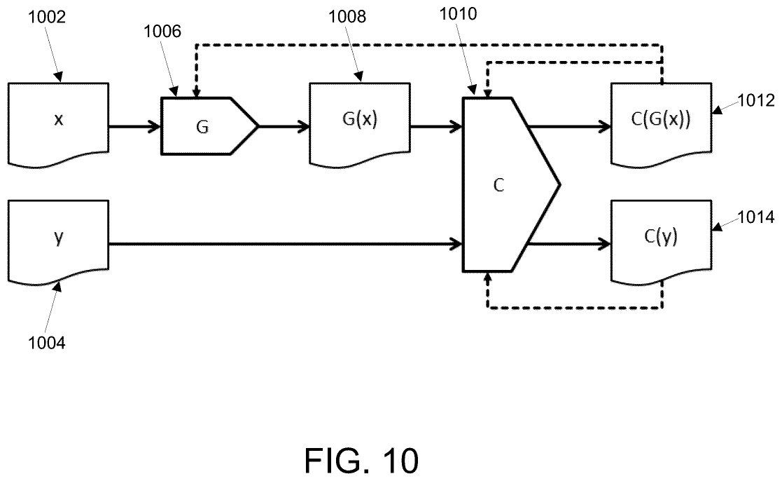

FIG. 10 shows a data flow diagram according to an embodiment for using a generative adversarial network for creating synthetic output data G(x) 1008 based on input data x 1002 that is indistinguishable from real output data y 1004, in accordance with one or more embodiments. The synthetic output data G(x) 1008 has the same structure as the real output data y 1004, but its content is not derived from real world data.

The generative adversarial network comprises a generator function G 1006 and a classifier function C 1010 which are trained jointly. The task of the generator function G 1006 is to provide realistic synthetic output data G(x) 1008 based on input data x 1002, and the task of the classifier function C 1010 is to distinguish between real output data y 1004 and synthetic output data G(x) 1008. In particular, the output of the classifier function C 1010 is a real number between 0 and 1 corresponding to the probability of the input value being real data, so that an ideal classifier function would calculate an output value of C(y) 1014=1 for real data y 1004 and C(G(x)) 1012=0 for synthetic data G(x) 1008.

Within the training process, parameters of the generator function G 1006 are adapted so that the synthetic output data G(x) 1008 has the same characteristics as real output data y 1004, so that the classifier function C 1010 cannot distinguish between real and synthetic data anymore. At the same time, parameters of the classifier function C 1010 are adapted so that it distinguishes between real and synthetic data in the best possible way. Here, the training relies on pairs comprising input data x 1002 and the corresponding real output data y 1004. Within a single training step, the generator function G 1006 is applied to the input data x 1002 for generating synthetic output data G(x) 1008. Furthermore, the classifier function C 1010 is applied to the real output data y 1004 for generating a first classification result C(y) 1014. Additionally, the classifier function C 1010 is applied to the synthetic output data G(x) 1008 for generating a second classification result C(G(x)) 1012.

Adapting the parameters of the generative function G 1006 and the classifier function C 1010 is based on minimizing a cost function by using the backpropagation algorithm, respectively. In this embodiment, the cost function K_{C} for the classifier function C 1010 is K_{C} ∝-BCE (C(y), 1)-BCE (C(G(x), 0), wherein BCE denotes the binary cross entropy defined as BCE (z, z′)=z′·log(z)+(1−z′)·log (1−z). By using this cost function, both wrongly classifying real output data as synthetic (indicated by C(y)≈0) and wrongly classifying synthetic output data as real (indicated as C(G(x)) 1012≈1) increases the cost function K_{C} to be minimized. Furthermore, the cost function K_{G} for the generator function G 1006 is K_{G} ∝-BCE (C(G(x), 1)=−log (C(G(x). By using this cost function, correctly classified synthetic output data (indicated as C(G(x)) 1012=0) leads to an increase of the cost function K_{G} to be minimized.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1102 that may be used to implement systems, apparatuses, and methods described herein is depicted in FIG. 11. Computer 1102 includes a processor 1104 operatively coupled to a data storage device 1112 and a memory 1110. Processor 1104 controls the overall operation of computer 1102 by executing computer program instructions that define such operations. The computer program instructions may be stored in data storage device 1112, or other computer readable medium, and loaded into memory 1110 when execution of the computer program instructions is desired. Thus, the method and workflow steps or functions of FIGS. 1-3 can be defined by the computer program instructions stored in memory 1110 and/or data storage device 1112 and controlled by processor 1104 executing the computer program instructions. For example, the computer program instructions can be implemented as computer executable code programmed by one skilled in the art to perform the method and workflow steps or functions of FIGS. 1-3. Accordingly, by executing the computer program instructions, the processor 1104 executes the method and workflow steps or functions of FIGS. 1-3. Computer 1102 may also include one or more network interfaces 1106 for communicating with other devices via a network. Computer 1102 may also include one or more input/output devices 1108 that enable user interaction with computer 1102 (e.g., display, keyboard, mouse, speakers, buttons, etc.).

Processor 1104 may include both general and special purpose microprocessors, and may be the sole processor or one of multiple processors of computer 1102. Processor 1104 may include one or more central processing units (CPUs), for example. Processor 1104, data storage device 1112, and/or memory 1110 may include, be supplemented by, or incorporated in, one or more application-specific integrated circuits (ASICs) and/or one or more field programmable gate arrays (FPGAs).

Data storage device 1112 and memory 1110 each include a tangible non-transitory computer readable storage medium. Data storage device 1112, and memory 1110, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1108 may include peripherals, such as a printer, scanner, display screen, etc. For example, input/output devices 1108 may include a display device such as a cathode ray tube (CRT) or liquid crystal display (LCD) monitor for displaying information to the user, a keyboard, and a pointing device such as a mouse or a trackball by which the user can provide input to computer 1102.

An image acquisition device 1114 can be connected to the computer 1102 to input image data (e.g., medical images) to the computer 1102. It is possible to implement the image acquisition device 1114 and the computer 1102 as one device. It is also possible that the image acquisition device 1114 and the computer 1102 communicate wirelessly through a network. In a possible embodiment, the computer 1102 can be located remotely with respect to the image acquisition device 1114.

Any or all of the systems, apparatuses, and methods discussed herein may be implemented using one or more computers such as computer 1102.

One skilled in the art will recognize that an implementation of an actual computer or computer system may have other structures and may contain other components as well, and that FIG. 11 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:
Illustrative embodiment 1. A computer-implemented method comprising: receiving 1) one or more input medical images of a patient and 2) one or more first image acquisition parameters associated with the one or more input medical images; generating one or more synthetic medical images associated with one or more second image acquisition parameters, the one or more synthetic medical images generated from at least one of the one or more input medical images using one or more machine learning based generator networks based on the one or more first image acquisition parameters; performing a medical imaging analysis task using a machine learning based task network based on the one or more synthetic medical images; and outputting results of the medical imaging analysis task.
Illustrative embodiment 2. The computer-implemented method of illustrative embodiment 1, wherein the one or more first image acquisition parameters and the one or more second image acquisition parameters comprise b-values.
Illustrative embodiment 3. The computer-implemented method of any one of illustrative embodiments 1-2, wherein the one or more first image acquisition parameters and the one or more second image acquisition parameters comprise at least one of field strength, signal-to-noise ratio, sequence selection, or a number of averages.
Illustrative embodiment 4. The computer-implemented method of any one of illustrative embodiments 1-3, wherein the one or more machine learning based generator networks have a same architecture with different parameters.
Illustrative embodiment 5. The computer-implemented method of any one of illustrative embodiments 1-4, wherein the one or more first image acquisition parameters are out-of-domain of training data on which the machine learning based task network was trained and the one or more second image acquisition parameters are in-domain of the training data on which the machine learning based task network was trained.
Illustrative embodiment 6. The computer-implemented method of any one of illustrative embodiments 1-5, wherein performing a medical imaging analysis task using a machine learning based task network based on the one or more synthetic medical images comprises: performing the medical imaging analysis task further based on remaining images of the one or more input medical images, the remaining images remaining from the at least one of the one or more input medical images from which the one or more synthetic medical images are generated.
Illustrative embodiment 7. The computer-implemented method of illustrative embodiment 6, wherein performing a medical imaging analysis task using a machine learning based task network based on the one or more synthetic medical images comprises: concatenating the one or more synthetic medical images with the remaining images of the one or more input medical images; and performing the medical imaging analysis task based on the concatenated images.
Illustrative embodiment 8. The computer-implemented method of any one of illustrative embodiments 1-7, wherein the one or more input medical images comprise a T2-weighted image, a diffusion-weighted imaging image, an apparent diffusion coefficient images, and an anatomical mask.
Illustrative embodiment 9. The computer-implemented method of any one of illustrative embodiments 1-8, wherein the medical imaging analysis task comprises prostate cancer detection.
Illustrative embodiment 10. An apparatus comprising: means for receiving 1) one or more input medical images of a patient and 2) one or more first image acquisition parameters associated with the one or more input medical images; means for generating one or more synthetic medical images associated with one or more second image acquisition parameters, the one or more synthetic medical images generated from at least one of the one or more input medical images using one or more machine learning based generator networks based on the one or more first image acquisition parameters; means for performing a medical imaging analysis task using a machine learning based task network based on the one or more synthetic medical images; and means for outputting results of the medical imaging analysis task.
Illustrative embodiment 11. The apparatus of illustrative embodiment 10, wherein the one or more first image acquisition parameters and the one or more second image acquisition parameters comprise b-values.
Illustrative embodiment 12. The apparatus of any one of illustrative embodiments 10-11, wherein the one or more first image acquisition parameters and the one or more second image acquisition parameters comprise at least one of field strength, signal-to-noise ratio, sequence selection, or a number of averages.
Illustrative embodiment 13. The apparatus of any one of illustrative embodiments 10-12, wherein the one or more machine learning based generator networks have a same architecture with different parameters.
Illustrative embodiment 14. The apparatus of any one of illustrative embodiments 10-13, wherein the one or more first image acquisition parameters are out-of-domain of training data on which the machine learning based task network was trained and the one or more second image acquisition parameters are in-domain of the training data on which the machine learning based task network was trained.
Illustrative embodiment 15. A non-transitory computer-readable storage medium comprising instructions which, when executed by a computer, cause the computer to carry out operations comprising: receiving 1) one or more input medical images of a patient and 2) one or more first image acquisition parameters associated with the one or more input medical images; generating one or more synthetic medical images associated with one or more second image acquisition parameters, the one or more synthetic medical images generated from at least one of the one or more input medical images using one or more machine learning based generator networks based on the one or more first image acquisition parameters; performing a medical imaging analysis task using a machine learning based task network based on the one or more synthetic medical images; and outputting results of the medical imaging analysis task.
Illustrative embodiment 16. The non-transitory computer-readable storage medium of illustrative embodiment 15, wherein the one or more first image acquisition parameters and the one or more second image acquisition parameters comprise b-values.
Illustrative embodiment 17. The non-transitory computer-readable storage medium of any one of illustrative embodiments 15-16, wherein performing a medical imaging analysis task using a machine learning based task network based on the one or more synthetic medical images comprises: performing the medical imaging analysis task further based on remaining images of the one or more input medical images, the remaining images remaining from the at least one of the one or more input medical images from which the one or more synthetic medical images are generated.
Illustrative embodiment 18. The non-transitory computer-readable storage medium of any one of illustrative embodiments 15-17, wherein performing a medical imaging analysis task using a machine learning based task network based on the one or more synthetic medical images comprises: concatenating the one or more synthetic medical images with the remaining images of the one or more input medical images; and performing the medical imaging analysis task based on the concatenated images.
Illustrative embodiment 19. The non-transitory computer-readable storage medium of any one of illustrative embodiments 15-18, wherein the one or more input medical images comprise a T2-weighted image, a diffusion-weighted imaging image, an apparent diffusion coefficient images, and an anatomical mask.
Illustrative embodiment 20. The non-transitory computer-readable storage medium of any one of illustrative embodiments 15-19, wherein the medical imaging analysis task comprises prostate cancer detection.

1. A computer-implemented method comprising:
receiving 1) one or more input medical images of a patient and 2) one or more first image acquisition parameters associated with the one or more input medical images;
generating one or more synthetic medical images associated with one or more second image acquisition parameters, the one or more synthetic medical images generated from at least one of the one or more input medical images using one or more machine learning based generator networks based on the one or more first image acquisition parameters;
performing a medical imaging analysis task using a machine learning based task network based on the one or more synthetic medical images; and
outputting results of the medical imaging analysis task.

2. The computer-implemented method of claim 1, wherein the one or more first image acquisition parameters and the one or more second image acquisition parameters comprise b-values.

3. The computer-implemented method of claim 1, wherein the one or more first image acquisition parameters and the one or more second image acquisition parameters comprise at least one of field strength, signal-to-noise ratio, sequence selection, or a number of averages.

4. The computer-implemented method of claim 1, wherein the one or more machine learning based generator networks have a same architecture with different parameters.

5. The computer-implemented method of claim 1, wherein the one or more first image acquisition parameters are out-of-domain of training data on which the machine learning based task network was trained and the one or more second image acquisition parameters are in-domain of the training data on which the machine learning based task network was trained.

6. The computer-implemented method of claim 1, wherein performing a medical imaging analysis task using a machine learning based task network based on the one or more synthetic medical images comprises:
performing the medical imaging analysis task further based on remaining images of the one or more input medical images, the remaining images remaining from the at least one of the one or more input medical images from which the one or more synthetic medical images are generated.

7. The computer-implemented method of claim 6, wherein performing a medical imaging analysis task using a machine learning based task network based on the one or more synthetic medical images comprises:
concatenating the one or more synthetic medical images with the remaining images of the one or more input medical images; and
performing the medical imaging analysis task based on the concatenated images.

8. The computer-implemented method of claim 1, wherein the one or more input medical images comprise a T2-weighted image, a diffusion-weighted imaging image, an apparent diffusion coefficient images, and an anatomical mask.

9. The computer-implemented method of claim 1, wherein the medical imaging analysis task comprises prostate cancer detection.

10. An apparatus comprising:
means for receiving 1) one or more input medical images of a patient and 2) one or more first image acquisition parameters associated with the one or more input medical images;
means for generating one or more synthetic medical images associated with one or more second image acquisition parameters, the one or more synthetic medical images generated from at least one of the one or more input medical images using one or more machine learning based generator networks based on the one or more first image acquisition parameters;
means for performing a medical imaging analysis task using a machine learning based task network based on the one or more synthetic medical images; and
means for outputting results of the medical imaging analysis task.

11. The apparatus of claim 10, wherein the one or more first image acquisition parameters and the one or more second image acquisition parameters comprise b-values.

12. The apparatus of claim 10, wherein the one or more first image acquisition parameters and the one or more second image acquisition parameters comprise at least one of field strength, signal-to-noise ratio, sequence selection, or a number of averages.

13. The apparatus of claim 10, wherein the one or more machine learning based generator networks have a same architecture with different parameters.

14. The apparatus of claim 10, wherein the one or more first image acquisition parameters are out-of-domain of training data on which the machine learning based task network was trained and the one or more second image acquisition parameters are in-domain of the training data on which the machine learning based task network was trained.

15. A non-transitory computer-readable storage medium comprising instructions which, when executed by a computer, cause the computer to carry out operations comprising:
receiving 1) one or more input medical images of a patient and 2) one or more first image acquisition parameters associated with the one or more input medical images;
generating one or more synthetic medical images associated with one or more second image acquisition parameters, the one or more synthetic medical images generated from at least one of the one or more input medical images using one or more machine learning based generator networks based on the one or more first image acquisition parameters;
performing a medical imaging analysis task using a machine learning based task network based on the one or more synthetic medical images; and
outputting results of the medical imaging analysis task.

16. The non-transitory computer-readable storage medium of claim 15, wherein the one or more first image acquisition parameters and the one or more second image acquisition parameters comprise b-values.

17. The non-transitory computer-readable storage medium of claim 15, wherein performing a medical imaging analysis task using a machine learning based task network based on the one or more synthetic medical images comprises:
performing the medical imaging analysis task further based on remaining images of the one or more input medical images, the remaining images remaining from the at least one of the one or more input medical images from which the one or more synthetic medical images are generated.

18. The non-transitory computer-readable storage medium of claim 17, wherein performing a medical imaging analysis task using a machine learning based task network based on the one or more synthetic medical images comprises:
concatenating the one or more synthetic medical images with the remaining images of the one or more input medical images; and
performing the medical imaging analysis task based on the concatenated images.

19. The non-transitory computer-readable storage medium of claim 15, wherein the one or more input medical images comprise a T2-weighted image, a diffusion-weighted imaging image, an apparent diffusion coefficient images, and an anatomical mask.

20. The non-transitory computer-readable storage medium of claim 15, wherein the medical imaging analysis task comprises prostate cancer detection.
